(19) United States
(12) Patent Application Publication
(10) Pub. No.: US 2012/0052501 A1
(43) Pub. Date: Mar. 1, 2012

(54) KIT FOR DETECTING HTLV STRAINS AND USE THEREOF

(75) Inventor: Jun LI, Baltimore, MD (US)

(73) Assignee: SAMSUNG TECHWIN CO., LTD., Changwon-city (KR)

(21) Appl. No.: 13/160,813

(22) Filed: Jun. 15, 2011

Related U.S. Application Data
(60) Provisional application No. 61/378,066, filed on Aug. 30, 2010.

(51) Int. Cl. C12Q 1/68; C07H 21/00

(52) U.S. Cl. 435/6.12; 536/24.33

(57) ABSTRACT

Methods and kits are described for the rapid and quantitative real-time PCR detection of HTLV nucleic acid sequences in biological samples. The procedure promises to facilitate the high throughput detection of HTLV in a cost effective and reliable manner.

### CROSS-REFERENCE TO RELATED APPLICATIONS

This application claims priority from U.S. Provisional Patent Application No. 61/378,066, filed on Aug. 30, 2010, the contents of which are hereby incorporated by reference in their entirety.

### FIELD

The present invention relates to methods and a kit of reagents for the real-time PCR detection of Human T-cell Lymphotropic Virus (HTLV).

### BACKGROUND

Human T-cell Lymphotropic Virus (HTLV) is a retrovirus that causes lymphoma, adult T-cell leukemia and is suspected of being involved in the etiology of a number of serious diseases including rheumatoid arthritis, Sjoegren syndrome, systemic lupus erythematosus, and uveitis. An estimated 20,000,000 people are estimated to be infected with HTLV worldwide.

One of the most widely used techniques to detect viral gene expression exploits first-strand cDNA of mRNA sequence(s) as a template for PCR amplification. The ability to measure the kinetics of a PCR reaction in combination with reverse transcriptase-PCR techniques promises to facilitate the accurate and precise measurement of viral target RNA sequences with the requisite level of sensitivity.

In particular, fluorescent dual-labeled hybridization probe technologies, such as the “CATACLEAVE™ endonuclease assay (described in detail in U.S. Pat. No. 5,763,181; see FIG. 1), permit the detection of reverse transcriptase-PCR amplification in real time. Detection of target sequences is achieved by including a CATACLEAVE™ probe in the amplification reaction together with RNase H. The CATACLEAVE™ probe, which is complementary to a target sequence within the reverse transcriptase—PCR amplification product, has a chimeric structure comprising an RNA sequence and a DNA sequence, and is flanked at its 5′ and 3′ ends by a detectable marker, for example FRET pair labeled DNA sequences. The proximity of the FRET pair's fluorescent label to the quencher precludes fluorescence of the intact probe. Upon annealing of the probe to the reverse transcriptase—PCR product a RNA: DNA duplex is generated that can be cleaved by RNase H present in the reaction mixture. Cleavage within the RNA portion of the annealed probe results in the separation of the fluorescent label from the quencher and a subsequent emission of fluorescence.

### SUMMARY

Methods and kits are described for the rapid and quantitative real-time PCR detection of HTLV nucleic acid sequences. The procedure promises to facilitate the high throughput detection of HTLV in a cost effective and reliable manner.

In one embodiment, a kit is described for the real-time PCR detection of HTLV strains comprising one or more of the following primer-probe sets:

a primer-probe set comprising a forward amplification primer comprising at least 10 consecutive nucleotides selected from the nucleotide sequence of SEQ ID NO:1 and a reverse amplification primer comprising at least 10 consecutive nucleotides selected from the nucleotide sequence of SEQ ID NO: 2 and a probe having the nucleotide sequence of SEQ ID NO: 9;

a primer-probe set comprising a forward amplification primer comprising at least 10 consecutive nucleotides selected from the nucleotide sequence of SEQ ID NO:3 and a reverse amplification primer comprising at least 10 consecutive nucleotides selected from the nucleotide sequence of SEQ ID NO: 4 and a probe having the nucleotide sequence of SEQ ID NO: 9;

a primer-probe set comprising a forward amplification primer comprising at least 10 consecutive nucleotides selected from the nucleotide sequence of SEQ ID NO:10 and a reverse amplification primer comprising at least 10 consecutive nucleotides selected from the nucleotide sequence of SEQ ID NO: 11, and a probe having the nucleotide sequence of SEQ ID NO: 14, 15, 16, 17 or 18;

a primer-probe set comprising a forward amplification primer comprising at least 10 consecutive nucleotides selected from the nucleotide sequence of SEQ ID NO:10 and a reverse amplification primer comprising at least 10 consecutive nucleotides selected from the nucleotide sequence of SEQ ID NO: 12, and a probe having the nucleotide sequence of SEQ ID NO: 14, 15, 16, 17 or 18;

a primer-probe set comprising a forward amplification primer comprising at least 10 consecutive nucleotides selected from the nucleotide sequence of SEQ ID NO:10 and a reverse amplification primer comprising at least 10 consecutive nucleotides selected from the nucleotide sequence of SEQ ID NO: 13, and a probe having the nucleotide sequence of SEQ ID NO: 14, 15, 16, 17 or 18;

a primer-probe set comprising a forward amplification primer comprising at least 10 consecutive nucleotides selected from the nucleotide sequence of SEQ ID NO:5 and a reverse amplification primer comprising at least 10 consecutive nucleotides selected from the nucleotide sequence of SEQ ID NO: 6 and a probe having the nucleotide sequence of SEQ ID NO: 9;

a primer-probe set comprising a forward amplification primer comprising at least 10 consecutive nucleotides selected from the nucleotide sequence of SEQ ID NO:7 and a reverse amplification primer comprising at least 10 consecutive nucleotides selected from the nucleotide sequence of SEQ ID NO: 8 and a probe having the nucleotide sequence of SEQ ID NO: 9; and

a primer-probe set comprising a forward amplification primer comprising at least 10 consecutive nucleotides selected from the nucleotide sequence of SEQ ID NO:19 and a reverse amplification primer comprising at least 10 consecutive nucleotides selected from the nucleotide sequence of SEQ ID NO: 20 and a probe having the nucleotide sequence of SEQ ID NO: 21, 22, 23, 24, 25.

The probe can be labeled with a fluorescent label such as a FRET pair. The probe can be linked to a solid support.

The kit can include positive internal and negative controls, uracil-N-glycosylase, an amplification buffer, an amplifying polymerase activity such as a thermostable DNA polymerase, a reverse transcriptase activity for the reverse transcription of a target HTLV RNA sequence to produce a target cDNA sequence, an RNase H activity such as an enzymatic activity of a thermostable RNase H or a hot start RNase H activity.

The 5′ end of each probe can be labeled with a fluorescent marker selected from the group consisting of FAM, VIC, TET, JOE, HEX, CY3, CY5, ROX, RED610, TEXAS RED, RED670, and NED, and the 3′ end of each probe can be labeled with a fluorescence quencher selected from the group consisting of 6-TAMRA, BHQ-1,2,3, and a molecular groove binding non-fluorescence quencher (MGBNFQ).

The HTLV strains can be HTLV-I, HTLV-II, HTLV-III, or HTLV-IV.

In another embodiment, a method is disclosed for the real-time detection of HTLV strains in a sample, comprising the steps of providing a sample to be tested for the presence of a HTLV gene target DNA, providing a pair of amplification primers that can anneal to the HTML gene target DNA, wherein the pair of amplification primers is selected from one of the above-described primer-probe sets, providing a probe of the primer-probe set comprising a detectable label and DNA and RNA nucleic acid sequences that are substantially complimentary to the HTLV target DNA sequence, amplifying a PCR fragment between the forward and reverse amplification primers in the presence of an amplifying polymerase activity, amplification buffer; an RNase H activity and the probe under conditions where the RNA sequences within the probe can form a RNA:DNA heteroduplex with the complimentary DNA sequences in the PCR fragment of the HTLV target DNA and detecting a real-time increase in the emission of a signal from the label on the probe, wherein the increase in signal indicates the presence of the HTLV target nucleic acid sequences in the sample.

In another embodiment, a method is disclosed for the real-time PCR detection of HTLV in a sample, comprising the steps of providing a sample to be tested for the presence of a HTLV target RNA, providing a pair of forward and reverse amplification primers that can anneal to the HTLV target nucleic acid sequence, wherein the pair of amplification primers is selected from one of the primer-probe sets described above, providing a probe comprising a detectable label and DNA and RNA nucleic acid sequences that are substantially complimentary to the cDNA of the HTLV target RNA, reverse transcribing the HTLV target RNA in the presence of a reverse transcriptase activity and the reverse amplification primer to produce a target cDNA sequence, amplifying a PCR fragment between the forward and reverse amplification primers in the presence of the target cDNA sequence, an amplifying polymerase activity, an amplification buffer; an RNase H activity and the probe under conditions where the RNA sequences within the probe can form a RNA:DNA heteroduplex with complimentary sequences in the PCR fragment, and detecting a real-time increase in the emission of a signal from the label on the probe, wherein the increase in signal indicates the presence of the HTLV target RNA sequences in the sample.

The real-time increase in the emission of the signal from the label on the probe can result from the RNase H cleavage of the heteroduplex formed between the probe and one of the strands of the PCR fragment.

The DNA and RNA sequences of the probe can be covalently linked. The probe can be can be labeled with a fluorescent label or a FRET pair.

The amplification buffer can be a Tris-acetate buffer. The PCR fragment can be linked to a solid support.

The amplifying polymerase activity can be an activity of a thermostable DNA polymerase.

The RNase H activity can be the activity of a thermostable RNase H. The RNase H activity can be a hot start RNase H activity. The nucleic acid within the sample can be pre-treated with uracil-N-glycosylase that can be inactivated prior to PCR amplification.

The HTLV target DNA can be a HTLV-I, HTLV-II, HTLV-III, and HTLV-IV target DNA.

The previously described embodiments have many advantages, including the ability to detect HTLV nucleic acid sequences in real-time. The detection method is fast, accurate and suitable for high throughput applications. Convenient, user-friendly and reliable diagnostic kits are also described for the detection of different HTLV strains.

### BRIEF DESCRIPTION OF THE DRAWINGS

The skilled artisan will understand that the drawings, described below, are for illustration purposes only. The figures are not intended to limit the scope of the teachings in any way.

FIG. 1 shows amplification curves obtained by real-time polymerase chain reaction (PCR) of HTLV-1 using a kit according to an embodiment of the present invention.

FIG. 2 shows amplification curves obtained by real-time polymerase chain reaction (PCR) of HTLV-2 using a kit according to an embodiment of the present invention.

FIGS. 3(A)-3(D) show detection of HTLV-1 and HTLV-2 using a multiplex real-time PCR assay. A: Detection of 10 and 10⁶ copies of HTLV-1 with Probe SEQ ID. 17; B: No fluorescence signals of HTLV-1 by Probe SEQ ID. 25; C: No fluorescence signals of HTLV-2 by Probe SEQ ID. 17; and D: Detection of 10 and 10⁶ copies of HTLV-2 with Probe SEQ ID. 25.

### DETAILED DESCRIPTION

The practice of the invention employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

### HTLV Primer Selection

A person of skill in the art will know how to design PCR primers flanking a HTLV target nucleic acid sequence of interest. Synthesized oligos are typically between 20 and 26 base pairs in length with a melting temperature, T_{M} of around 55 degrees.

In a preferred embodiment, HTLV primer sequences are selected for their inability to form primer dimers in a standard PCR reaction. A “primer dimer” is a potential by-product in PCR, that consists of primer molecules that have partially hybridized to each other because of strings of complementary bases in the primers. As a result, the DNA polymerase amplifies the primer dimer, leading to competition for PCR reagents, thus potentially inhibiting amplification of the DNA sequence targeted for PCR amplification. In real-time PCR, primer dimers may interfere with accurate quantification by reducing sensitivity.

A “target DNA or “target RNA”” or “target nucleic acid,” or “target nucleic acid sequence” refers to a HTLV nucleic acid sequence that is targeted by DNA amplification. A target nucleic acid sequence serves as a template for amplification in a PCR reaction or reverse transcriptase-PCR reaction. Target nucleic acid sequences may include both naturally occurring and synthetic molecules. Exemplary target nucleic acid sequences include, but are not limited to, HTLV genomic DNA or genomic RNA.

The “primer” used herein is a single-stranded oligonucleotide functioning as an origin of polymerization of template DNA under appropriate conditions (i.e., 4 types of different nucleoside triphosphates and polymerases) at a suitable temperature and in a suitable buffer solution. The length of the primer may vary according to various factors, for example, temperature and the use of the primer, but the primer generally has 15 to 30 nucleotides. Generally, a short primer may form a sufficiently stable hybrid complex with its template at a low temperature. The “forward primer” and “reverse primer” are primers respectively binding to a 3′ end and a 5′ end of a specific region of a template that is amplified by PCR. The sequence of the primer is not required to be completely complementary to a part of the sequence of the template. The primer may have sufficient complementarity to be hybridized with the template and perform intrinsic functions of the primer. Thus, a primer set according to an embodiment is not required to be completely complementary to the nucleotide sequence as a template. The primer set may have sufficient complementarity to be hybridized with the sequence and perform intrinsic functions of the primer. The primer may be designed based on the nucleotide sequence of a polynucleotide as a template, for example, using a program for designing primers (PRIMER 3 program). Meanwhile, a primer according to an embodiment may be hybridized or annealed to a part of a template to form a double-strand. Conditions for hybridizing nucleic acid suitable for forming the double-stranded structure are disclosed by Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985) For example, the primer may include at least 10 or at least 15 consecutive nucleotides of any one of the nucleotide sequences of SEQ ID NOS: 1 to 12. The primer may also be a nucleotide having any one of the nucleotide sequences of SEQ ID NOS: 1 to 12.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

### Nucleic Acid Template Preparation

In some embodiments, the sample comprises a purified nucleic acid template (e.g., mRNA, rRNA, and mixtures thereof).

In other embodiments, the sample may include cultured cells and animal or human blood, plasma, serum, sperm, or mucus, but is not limited thereto.

Procedures for the extraction and purification of nucleic acid from samples are well known in the art. For example, RNA can be isolated from cells using the TRIzol™ reagent (Invitrogen) extraction method. RNA quantity and quality is then determined using, for example, a Nanodrop™ spectrophotometer and an Agilent 2100 bioanalyzer (see also Peirson S N, Butler J N (2007). “RNA extraction from mammalian tissues” Methods Mol. Biol. 362: 315-27, Bird I M (2005) “Extraction of RNA from cells and tissue” Methods Mol. Med. 108: 139-48).

Exemplary methods of extracting nucleic acid from whole blood are taught by Casareale et al. (1992) (Improved blood sample processing for PCR Genome Res. (1992) 2: 149-153) and by U.S. Pat. No. 5,334,499.

In addition, several commercial kits are available for the isolation of viral nucleic acids from whole blood. Exemplary kits include, but are not limited to, QIAamp DNA Blood Mini Kit (Qiagen; Cat. No. 51104), MagNA Pure Compact Nucleic Acid Isolation Kit (Roche Applied Sciences; Cat. No. 03730964001), Stabilized Blood-to-CT™ Nucleic Acid Preparation Kit for qPCR (Invitrogen, Cat. No. 4449080) and GF-1 Viral Nucleic Acid Extraction Kit (GeneOn, Cat. No. RD05).

### PCR Amplification of HTLV Target Nucleic Acid Sequences

Once the primers are prepared, nucleic acid amplification can be accomplished by a variety of methods, including, but not limited to, the polymerase chain reaction (PCR), nucleic acid sequence based amplification (NASBA), ligase chain reaction (LCR), and rolling circle amplification (RCA). The polymerase chain reaction (PCR) is the method most commonly used to amplify specific target DNA sequences.

“Polymerase chain reaction,” or “PCR,” generally refers to a method for amplification of a desired nucleotide sequence in vitro. Generally, the PCR process consists of introducing a molar excess of two or more extendable oligonucleotide primers to a reaction mixture comprising a sample having the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers.

The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference.

The term “sample” refers to any substance containing nucleic acid material.

As used herein, the term “PCR fragment” or “reverse transcriptase-PCR fragment” or “amplicon” refers to a polynucleotide molecule (or collectively the plurality of molecules) produced following the amplification of a particular target nucleic acid. A PCR fragment is typically, but not exclusively, a DNA PCR fragment. A PCR fragment can be single-stranded or double-stranded, or in a mixture thereof in any concentration ratio. A PCR fragment or RT-PCT can be about 100 to about 500 nt or more in length.

A “buffer” is a compound added to an amplification reaction which modifies the stability, activity, and/or longevity of one or more components of the amplification reaction by regulating the pH of the amplification reaction. The buffering agents of the invention are compatible with PCR amplification and site-specific RNase H cleavage activity. Certain buffering agents are well known in the art and include, but are not limited to, Tris, Tricine, MOPS (3-(N-morpholino)propanesulfonic acid), and HEPES (4-(2-hydroxyethyl)-1-piperazineethanesulfonic acid). In addition, PCR buffers may generally contain up to about 70 mM KCl and about 1.5 mM or higher MgCl₂, to about 50-200 μM each of nucleotides dATP, dCTP, dGTP and dTTP. The buffers of the invention may contain additivies to optimize efficient reverse transcriptase-PCR or PCR reaction.

The term “nucleotide,” as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR2 or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′-and 3′-4′-linked and other “locked” or “LNA”, bicyclic sugar modifications (see, e.g., PCT published application nos. WO 98/22489, WO 98/39352, and WO 99/14226; and U.S. Pat. Nos. 6,268,490 and 6,794,499).

An additive is a compound added to a composition which modifies the stability, activity, and/or longevity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, betaine, formamide, KCl, CaCl₂, MgOAc, MgCl₂, NaCl, NH₄OAc, NaI, Na(CO₃)₂, LiCl, MnOAc, NMP, trehalose, demethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll™, aurintricarboxylic acid, Tween 20, Tween 21, Tween 40, Tween 60, Tween 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, UNG, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. According to the invention, additives may be added to improve selectivity of primer annealing provided the additives do not interfere with the activity of RNase H.

As used herein, the term “thermostable,” as applied to an enzyme, refers to an enzyme that retains its biological activity at elevated temperatures (e.g., at 55° C. or higher), or retains its biological activity following repeated cycles of heating and cooling. Thermostable polynucleotide polymerases find particular use in PCR amplification reactions.

As used herein, an “amplifying polymerase activity” refers to an enzymatic activity that catalyzes the polymerization of deoxyribonucleotides. Generally, the enzyme will initiate synthesis at the 3′-end of the primer annealed to a nucleic acid template sequence, and will proceed toward the 5′ end of the template strand. In certain embodiments, an “amplifying polymerase activity” is a thermostable DNA polymerase.

As used herein, a thermostable polymerase is an enzyme that is relatively stable to heat and eliminates the need to add enzyme prior to each PCR cycle.

Non-limiting examples of thermostable DNA polymerases may include, but are not limited to, polymerases isolated from the thermophilic bacteria Thermus aquaticus (Taq polymerase), Thermus thermophilus (Tth polymerase), Thermococcus litoralis (Tli or VENT™ polymerase), Pyrococcus furiosus (Pfu or DEEPVENT™ polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME™ polymerase) (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction. In one embodiment, the thermostable polymerase may be Taq polymerase. Many variants of Taq polymerase with enhanced properties are known and include, but are not limited to, AmpliTaq™, AmpliTaq™, Stoffel fragment, SuperTaq™, SuperTaq™ plus, LA Taq™, LApro Taq™, and EX Taq ™. In another embodiment, the thermostable polymerase used in the multiplex amplification reaction of the invention is the AmpliTaq Stoffel fragment.

### Reverse Transcriptase-PCR Amplification of a HTLV RNA Target Nucleic Acid Sequence

One of the most widely used techniques to study gene expression exploits first-strand cDNA for mRNA sequence(s) as template for amplification by the PCR.

The term “reverse transcriptase activity” and “reverse transcription” refers to the enzymatic activity of a class of polymerases characterized as RNA-dependent DNA polymerases that can synthesize a DNA strand (i.e., complementary DNA, cDNA) utilizing an RNA strand as a template.

“Reverse transcriptase-PCR” of “RNA PCR” is a PCR reaction that uses RNA template and a reverse transcriptase, or an enzyme having reverse transcriptase activity, to first generate a single stranded DNA molecule prior to the multiple cycles of DNA-dependent DNA polymerase primer elongation. Multiplex PCR refers to PCR reactions that produce more than one amplified product in a single reaction, typically by the inclusion of more than two primers in a single reaction.

Exemplary reverse transcriptases include, but are not limited to, the Moloney murine leukemia virus (M-MLV) RT as described in U.S. Pat. No. 4,943,531, a mutant form of M-MLV-RT lacking RNase H activity as described in U.S. Pat. No. 5,405,776, bovine leukemia virus (BLV) RT, Rous sarcoma virus (RSV) RT, Avian Myeloblastosis Virus (AMV) RT and reverse transcriptases disclosed in U.S. Pat. No. 7,883,871.

The reverse transcriptase-PCR procedure, carried out as either an end-point or real-time assay, involves two separate molecular syntheses: (i) the synthesis of HTLV cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” reverse transcriptase-PCR methods use a common or compromised buffer for reverse transcriptase and Taq DNA polymerase activities. In one version, the annealing of a reverse primer is a separate step preceding the addition of enzymes, which are then added to the single reaction vessel. In another version, the reverse transcriptase activity is a component of the thermostable Tth DNA polymerase Annealing and cDNA synthesis are performed in the presence of Mn²⁺ then PCR is carried out in the presence of Mg²⁺ after the removal of Mn²⁺ by a chelating agent. Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMV RT and Taq DNA Polymerase wherein the initial 65° C. RNA denaturation step may be omitted.

In certain embodiments, one or more primers may be labeled. As used herein, “label,” “detectable label,” or “marker,” or “detectable marker,” which are interchangeably used in the specification, refers to any chemical moiety attached to a nucleotide, nucleotide polymer, or nucleic acid binding factor, wherein the attachment may be covalent or non-covalent. Preferably, the label is detectable and renders the nucleotide or nucleotide polymer detectable to the practitioner of the invention. Detectable labels include luminescent molecules, chemiluminescent molecules, fluorochromes, fluorescent quenching agents, colored molecules, radioisotopes or scintillants. Detectable labels also include any useful linker molecule (such as biotin, avidin, streptavidin, HRP, protein A, protein G, antibodies or fragments thereof, Grb2, polyhistidine, Ni²⁺, FLAG tags, myc tags), heavy metals, enzymes (examples include alkaline phosphatase, peroxidase and luciferase), electron donors/acceptors, acridinium esters, dyes and calorimetric substrates. It is also envisioned that a change in mass may be considered a detectable label, as is the case of surface plasmon resonance detection. The skilled artisan would readily recognize useful detectable labels that are not mentioned above, which may be employed in the operation of the present invention.

One step reverse transcriptase-PCR provides several advantages over uncoupled reverse transcriptase-PCR. One step reverse transcriptase-PCR requires less handling of the reaction mixture reagents and nucleic acid products than uncoupled reverse transcriptase-PCR (e.g., opening of the reaction tube for component or enzyme addition in between the two reaction steps), and is therefore less labor intensive, reducing the required number of person hours. One step reverse transcriptase-PCR also requires less sample, and reduces the risk of contamination. The sensitivity and specificity of one-step reverse transcriptase-PCR has proven well suited for studying expression levels of one to several genes in a given sample or the detection of pathogen RNA. Typically, this procedure has been limited to use of gene-specific primers to initiate cDNA synthesis.

The ability to measure the kinetics of a PCR reaction by on-line detection in combination with these reverse transcriptase-PCR techniques has enabled accurate and precise measurement of RNA sequences with high sensitivity. This has become possible by detecting the reverse transcriptase-PCR product through fluorescence monitoring and measurement of PCR product during the amplification process by fluorescent dual-labeled hybridization probe technologies, such as the 5′ fluorogenic nuclease assay (“TaqMan™”) or endonuclease assay (“CataCleave™”), discussed below.

### Real-Time PCR Detection of HTLV Target Nucleic Acid Sequences Using a Catacleave Probe

Post amplification amplicon detection can be both laborious and time consuming. Real-time methods have been developed to monitor amplification during the PCR process. These methods typically employ fluorescently labeled probes that bind to the newly synthesized DNA or dyes whose fluorescence emission is increased when intercalated into double stranded DNA. Real-time detection methodologies are applicable to PCR detection of HTLV sequences in genomic DNA or genomic RNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red.) Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons(e.g., U.S. Pat. No. 5,925,517), TaqMan™ probes (e.g., U.S. Pat. Nos. 5,210,015 and 5,487,972), and CataCleave™ probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded the distance between the donor and acceptor chromophores increases such that FRET is reversed and donor emission can be monitored using specialized instrumentation. TaqMan™ and CataCleave™ technologies differ from the molecular beacon in that the FRET probes employed are cleaved such that the donor and acceptor chromophores become sufficiently separated to reverse FRET.

In certain embodiments, the probe is designed to hybridize with the template target nucleic acid sequence under stringent conditions that are known in the art. The “stringent conditions” are disclosed, for example, in Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985), and may be determined by controlling temperature, ionic strength (concentration of a buffer solution), and the existence of a compound such as an organic solvent. For example, the stringent conditions may be obtained by a) washing with a 0.015 M sodium chloride/0.0015 M sodium citrate/0.1% sodium dodecyl sulfate solution at 50° C., or b) hybridizing in a hybridization buffer solution including 50% formamide, 2×SSC and 10% dextran sulfate at 55° C. and washing with EDTA-containing 0.1×SSC at 55° C.

In other embodiments, the probe is substantially complementary to the HTLV target nucleic acid sequence.

As used herein, the term “substantially complementary” refers to two nucleic acid strands that are sufficiently complimentary in sequence to anneal and form a stable duplex. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence.

Accordingly, “substantially complementary” sequences can refer to sequences with base-pair complementarity of 100, 95, 90, 80, 75, 70, 60, 50 percent or less, or any number in between, in a double-stranded region.

TaqMan™ technology employs a single stranded oligonucleotide probe that is labeled at the 5′ end with a donor chromophore and at the 3′ end with an acceptor chromophore. The DNA polymerase used for amplification must contain a 5′->3′ exonuclease activity. The TaqMan™ probe binds to one strand of the amplicon at the same time that the primer binds. As the DNA polymerase extends the primer the polymerase will eventually encounter the bound TaqMan™ probe. At this time the exonuclease activity of the polymerase will sequentially degrade the TaqMan™ probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan™ works a specific amplicon can be detected only once for every cycle of PCR. Extension of the primer through the TaqMan™ target site generates a double stranded product that prevents further binding of TaqMan™ probes until the amplicon is denatured in the next PCR cycle.

U.S. Pat. No. 5,763,181, of which content is incorporated herein by reference, describes another real-time detection method (referred to as “CataCleave™”). CataCleave™ technology differs from TaqMan™ in that cleavage of the probe is accomplished by a second enzyme that does not have polymerase activity. The CataCleave™ probe has a sequence within the molecule which is a target of an endonuclease, such as, for example a restriction enzyme or RNase. In one example, the CataCleave™ probe has a chimeric structure where the 5′ and 3′ ends of the probe are constructed of DNA and the cleavage site contains RNA. The DNA sequence portions of the probe are labeled with a FRET pair either at the ends or internally. The PCR reaction includes an RNase H enzyme that will specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan™ probe and donor emission can be monitored. Cleavage and dissociation regenerates a site for further CataCleave™ binding. In this way it is possible for a single amplicon to serve as a target or multiple rounds of probe cleavage until the primer is extended through the CataCleave™ probe binding site.

### Labeling of a Catacleave Probe

The term “probe” comprises a polynucleotide that comprises a specific portion designed to hybridize in a sequence-specific manner with a complementary region of a specific nucleic acid sequence, e.g., a target nucleic acid sequence. In one embodiment, the oligonucleotide probe is in the range of 15-60 nucleotides in length. More preferably, the oligonucleotide probe is in the range of 18-30 nucleotides in length. The precise sequence and length of an oligonucleotide probe of the invention depends in part on the nature of the target polynucleotide to which it binds. The binding location and length may be varied to achieve appropriate annealing and melting properties for a particular embodiment. Guidance for making such design choices can be found in many of the references describing TaqMan™ assays or CataCleave™, described in U.S. Pat. Nos. 5,763,181, 6,787,304, and 7,112,422, of which contents are incorporated herein by reference.

In certain embodiments, the probe is “substantially complementary” to the target nucleic acid sequence.

As used herein, “label” or “detectable label” of the CataCleave probe refers to any label comprising a fluorochrome compound that is attached to the probe by covalent or non-covalent means.

As used herein, “fluorochrome” refers to a fluorescent compound that emits light upon excitation by light of a shorter wavelength than the light that is emitted. The term “fluorescent donor” or “fluorescence donor” refers to a fluorochrome that emits light that is measured in the assays described in the present invention. More specifically, a fluorescent donor provides energy that is absorbed by a fluorescence acceptor. The term “fluorescent acceptor” or “fluorescence acceptor” refers to either a second fluorochrome or a quenching molecule that absorbs light emitted from the fluorescence donor. The second fluorochrome absorbs the energy that is emitted from the fluorescence donor and emits light of longer wavelength than the light emitted by the fluorescence donor. The quenching molecule absorbs energy emitted by the fluorescence donor.

Any luminescent molecule, preferably a fluorochrome and/or fluorescent quencher may be used in the practice of this invention, including, for example, Alexa Fluor™ 350, Alexa Fluor™ 430, Alexa Fluor™ 488, Alexa Fluor™ 532, Alexa Fluor™ 546, Alexa Fluor™ 568, Alexa Fluor™ 594, Alexa Fluor™ 633, Alexa Fluor™ 647, Alexa Fluor™ 660, Alexa Fluor™ 680, 7-diethylaminocoumarin-3-carboxylic acid, Fluorescein, Oregon Green 488, Oregon Green 514, Tetramethylrhodamine, Rhodamine X, Texas Red dye, QSY 7, QSY33, Dabcyl, BODIPY FL, BODIPY 630/650, BODIPY 6501665, BODIPY TMR-X, BODIPY TR-X, Dialkylaminocoumarin, Cy5.5, Cy5, Cy3.5, Cy3, DTPA(Eu³⁺)-AMCA and TTHA(Eu³⁺)AMCA.

In one embodiment, the 3′ terminal nucleotide of the oligonucleotide probe is blocked or rendered incapable of extension by a nucleic acid polymerase. Such blocking is conveniently carried out by the attachment of a reporter or quencher molecule to the terminal 3′ position of the probe.

In one embodiment, reporter molecules are fluorescent organic dyes derivatized for attachment to the terminal 3′ or terminal 5′ ends of the probe via a linking moiety. Preferably, quencher molecules are also organic dyes, which may or may not be fluorescent, depending on the embodiment of the invention. For example, in a preferred embodiment of the invention, the quencher molecule is fluorescent. Generally whether the quencher molecule is fluorescent or simply releases the transferred energy from the reporter by non-radiative decay, the absorption band of the quencher should substantially overlap the fluorescent emission band of the reporter molecule. Non-fluorescent quencher molecules that absorb energy from excited reporter molecules, but which do not release the energy radiatively, are referred to in the application as chromogenic molecules.

Exemplary reporter-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl6-naphthalene sulfonate. Other dyes include 3-phenyl-7-isocyanatocoumarin, acridines, such as 9-isothiocyanatoacridine and acridine orange, N-(p-(2-benzoxazolyl)phenyl)maleimide, benzoxadiazoles, stilbenes, pyrenes, and the like.

In one embodiment, reporter and quencher molecules are selected from fluorescein and rhodamine dyes.

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.) Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

Rhodamine and fluorescein dyes are also conveniently attached to the 5′ hydroxyl of an oligonucleotide at the conclusion of solid phase synthesis by way of dyes derivatized with a phosphoramidite moiety, e.g., Woo et al., U.S. Pat. No. 5,231,191; and Hobbs, Jr., U.S. Pat. No. 4,997,928.

### Attachment of a Catacleave Probe to a Solid Support

In one embodiment, the oligonucleotide probe can be attached to a solid support. Different probes may be attached to the solid support and may be used to simultaneously detect different target sequences in a sample. Reporter molecules having different fluorescence wavelengths can be used on the different probes, thus enabling hybridization to the different probes to be separately detected.

Examples of preferred types of solid supports for immobilization of the oligonucleotide probe include controlled pore glass, glass plates, polystyrene, avidin coated polystyrene beads cellulose, nylon, acrylamide gel and activated dextran, controlled pore glass (CPG), glass plates and high cross-linked polystyrene. These solid supports are preferred for hybridization and diagnostic studies because of their chemical stability, ease of functionalization and well defined surface area. Solid supports such as controlled pore glass (500 Å, 1000 Å) and non-swelling high cross-linked polystyrene (1000 Å) are particularly preferred in view of their compatibility with oligonucleotide synthesis.

The oligonucleotide probe may be attached to the solid support in a variety of manners. For example, the probe may be attached to the solid support by attachment of the 3′ or 5′ terminal nucleotide of the probe to the solid support. However, the probe may be attached to the solid support by a linker which serves to distance the probe from the solid support. The linker is most preferably at least 30 atoms in length, more preferably at least 50 atoms in length.

Hybridization of a probe immobilized to a solid support generally requires that the probe be separated from the solid support by at least 30 atoms, more-preferably at least 50 atoms. In order to achieve this separation, the linker generally includes a spacer positioned between the linker and the 3′ nucleoside. For oligonucleotide synthesis, the linker arm is usually attached to the 3′-OH of the 3′ nucleoside by an ester linkage which can be cleaved with basic reagents to free the oligonucleotide from the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred because it is commercially available, soluble in both organic and aqueous media, easy to functionalize, and completely stable under oligonucleotide synthesis and post-synthesis conditions.

The linkages between the solid support, the linker and the probe are preferably not cleaved during removal of base protecting groups under basic conditions at high temperature. Examples of preferred linkages include carbamate and amide linkages. Immobilization of a probe is well known in the art and one skilled in the art may determine the immobilization conditions.

According to one embodiment of the method, the CataCleave™ probe is immobilized on a solid support. The CataCleave™ probe comprises a detectable label and DNA and RNA nucleic acid sequences, wherein the probe's RNA nucleic acid sequences are complementary to a selected region of a target DNA sequence and the probe's DNA nucleic acid sequences are substantially complementary to DNA sequences adjacent to the selected region of the target DNA sequence. The probe is then contacted with a sample of nucleic acids in the presence of RNase H and under conditions where the RNA sequences within the probe can form a RNA:DNA heteroduplex with the complementary DNA sequences in the PCR fragment. RNase H cleavage of the RNA sequences within the RNA:DNA heteroduplex results in a real-time increase in the emission of a signal from the label on the probe, wherein the increase in signal indicates the presence of the polymorphism in the target DNA.

Immobilization of the probe to the solid support also enables the target sequence hybridized to the probe to be readily isolated from the sample. In later steps, the isolated target sequence may be separated from the solid support and processed (e.g., purified, amplified) according to methods well known in the art depending on the particular needs of the researcher.

### RNase H Cleavage of the Catacleave™ Probe

RNase H hydrolyzes RNA in RNA-DNA hybrids. First identified in calf thymus, RNase H has subsequently been described in a variety of organisms. Indeed, RNase H activity appears to be ubiquitous in eukaryotes and bacteria. Although RNase Hs form a family of proteins of varying molecular weight and nucleolytic activity, substrate requirements appear to be similar for the various isotypes. For example, most RNase Hs studied to date function as endonucleases and require divalent cations (e.g., Mg²⁺, Mn²⁺) to produce cleavage products with 5′ phosphate and 3′ hydroxyl termini.

In prokaryotes, RNase H have been cloned and extensively characterized (see Crooke, et al., (1995) Biochem J, 312 (Pt 2), 599-608; Lima, et al., (1997) J Biol Chem, 272, 27513-27516; Lima, et al., (1997) Biochemistry, 36, 390-398; Lima, et al., (1997) J Biol Chem, 272, 18191-18199; Lima, et al., (2007) Mol Pharmacol, 71, 83-91; Lima, et al., (2007) Mol Pharmacol, 71, 73-82; Lima, et al., (2003) J Biol Chem, 278, 14906-14912; Lima, et al., (2003) J Biol Chem, 278, 49860-49867; Itaya, M., Proc. Natl. Acad. Sci. USA, 1990, 87, 8587-8591). For example, E. coli RNase HII is 213 amino acids in length whereas RNase HI is 155 amino acids long. E. coli RNase HII displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxyl end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have been defined based on differences in molecular weight, effects of divalent cations, sensitivity to sulfhydryl agents and immunological cross-reactivity (Busen et al., Eur. J. Biochem., 1977, 74, 203-208). RNase HI enzymes are reported to have molecular weights in the 68-90 kDa range, be activated by either Mn²⁺ or Mg²⁺ and be insensitive to sulfhydryl agents. In contrast, RNase H II enzymes have been reported to have molecular weights ranging from 31-45 kDa, to require Mg²⁺ to be highly sensitive to sulfhydryl agents and to be inhibited by Mn²⁺ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108)

An enzyme with RNase HII characteristics has also been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5-9. The enzyme requires Mg²⁺ and is inhibited by Mn²⁺ and n-ethyl maleimide. The products of cleavage reactions have 3′ hydroxyl and 5′ phosphate termini.

A detailed comparison of RNases from different species is reported in Ohtani N, Haruki M, Morikawa M, Kanaya S. J Biosci Bioeng. 1999; 88(1):12-9.

Examples of RNase H enzymes, which may be employed in the embodiments, also include, but are not limited to, thermostable RNase H enzymes isolated from thermophilic organisms such as Pyrococcus furiosus RNase HII, Pyrococcus horikoshi RNase HII, Thermococcus litoralis RNase HI, Thermus thermophilus RNase HI.

Other RNase H enzymes that may be employed in the embodiments are described in, for example, U.S. Pat. No. 7,422,888 to Uemori or the published U.S. Patent Application No. 2009/0325169 to Walder, the contents of which are incorporated herein by reference.

In one embodiment, an RNase H enzyme is a thermostable RNase H with 40%, 50%, 60%, 70%, 80%, 90%, 95% or 99% homology with the amino acid sequence of Pfu RNase HII (SEQ ID NO: 26), shown below.

| (SEQ ID NO: 26) | |
|---|---|
| MKIGGIDEAG RGPAIGPLVV ATVVVDEKNI EKLRNIGVKD SKQLTPHERK NLFSQITSIA | 60 |
| DDYKIVIVSP EEIDNRSGTM NELEVEKFAL ALNSLQIKPA LIYADAADVD ANRFASLIER | 120 |
| RLNYKAKIIA EHKADAKYPV VSAASILAKV VRDEEIEKLK KQYGDFGSGY PSDPKTKKWL | 180 |
| EEYYKKHNSF PPIVRRTWET VRKIEESIKA KKSQLTLDKF FKKP | |

The homology can be determined using, for example, a computer program DNASIS-Mac (Takara Shuzo), a computer algorithm FASTA (version 3.0; Pearson, W. R. et al., Pro. Natl. Acad. Sci., 85:2444-2448, 1988) or a computer algorithm BLAST (version 2.0, Altschul et al., Nucleic Acids Res. 25:3389-3402, 1997).

In another embodiment, an RNase H enzyme is a thermostable RNase H with at least one or more homology regions 1-4 corresponding to positions 5-20, 33-44, 132-150, and 158-173 of SEQ ID NO: 26.

HOMOLOGY REGION 1:
GIDEAG RGPAIGPLVV
(SEQ ID NO: 27; corresponding to positions
5-20 of SEQ ID NO: 26)
HOMOLOGY REGION 2:
LRNIGVKD SKQL
(SEQ ID NO: 28; corresponding to positions
33-44 of SEQ ID NO: 26)
HOMOLOGY REGION 3:
HKADAKYPV VSAASILAKV
(SEQ ID NO: 29; corresponding to positions
132-150 of SEQ ID NO: 26)
HOMOLOGY REGION 4:
KLK KQYGDFGSGY PSD
(SEQ ID NO: 30; corresponding to positions
158-173 of SEQ ID NO: 26)

In another embodiment, an RNase H enzyme is a thermostable RNase H with at least one of the homology regions having 50%, 60%. 70%, 80%, 90% sequence identity with a polypeptide sequence of SEQ ID NOs: 27, 28, 29 and 30.

The terms “sequence identity” as used herein refers to the extent that sequences are identical or functionally or structurally similar on a amino acid to amino acid basis over a window of comparison. Thus, a “percentage of sequence identity”, for example, can be calculated by comparing two optimally aligned sequences over the window of comparison, determining the number of positions at which the identical amino acid occurs in both sequences to yield the number of matched positions, dividing the number of matched positions by the total number of positions in the window of comparison (i.e., the window size), and multiplying the result by 100 to yield the percentage of sequence identity.

In certain embodiments, the RNase H can be modified to produce a hot start “inducible” RNase H.

The term “modified RNase H,” as used herein, can be an RNase H reversely coupled to or reversely bound to an inhibiting factor that causes the loss of the endonuclease activity of the RNase H. Release or decoupling of the inhibiting factor from the RNase H restores at least partial or full activity of the endonuclease activity of the RNase H. About 30-100% of its activity of an intact RNase H may be sufficient. The inhibiting factor may be a ligand or a chemical modification. The ligand can be an antibody, an aptamer, a receptor, a cofactor, or a chelating agent. The ligand can bind to the active site of the RNase H enzyme thereby inhibiting enzymatic activity or it can bind to a site remote from the RNase's active site. In some embodiment, the ligand may induce a conformational change. The chemical modification can be a crosslinking (for example, by formaldehyde) or acylation. The release or decoupling of the inhibiting factor from the RNase HII may be accomplished by heating a sample or a mixture containing the coupled RNase HII (inactive) to a temperature of about 65° C. to about 95° C. or higher, and/or lowering the pH of the mixture or sample to about 7.0 or lower.

As used herein, a hot start “inducible” RNase H activity refers to the herein described modified RNase H that has an endonuclease catalytic activity that can be regulated by association with a ligand. Under permissive conditions, the RNase H endonuclease catalytic activity is activated whereas at non-permissive conditions, this catalytic activity is inhibited. In some embodiments, the catalytic activity of a modified RNase H can be inhibited at temperature conducive for reverse transcription, i.e. about 42° C., and activated at more elevated temperatures found in PCR reactions, i.e. about 65° C. to 95° C. A modified RNase H with these characteristics is said to be “heat inducible.”

In other embodiments, the catalytic activity of a modified RNase H can be regulated by changing the pH of a solution containing the enzyme.

As used herein, a “hot start” enzyme composition refers to compositions having an enzymatic activity that is inhibited at non-permissive temperatures, i.e. from about 25° C. to about 45° C. and activated at temperatures compatible with a PCR reaction, e.g. about 55° C. to about 95° C. In certain embodiment, a “hot start” enzyme composition may have a ‘hot start’ RNase H and/or a ‘hot start’ thermostable DNA polymerase that are known in the art.

Crosslinking of RNase H enzymes can be performed using, for example, formaldehyde. In one embodiment, a thermostable RNase HII is subjected to controlled and limited crosslinking using formaldehyde. By heating an amplification reaction composition, which comprises the modified RNase HII in an active state, to a temperature of about 95° C. or higher for an extended time, for example about 15 minutes, the crosslinking is reversed and the RNase HII activity is restored.

In general, the lower the degree of crosslinking, the higher the endonuclease activity of the enzyme is after reversal of crosslinking. The degree of crosslinking may be controlled by varying the concentration of formaldehyde and the duration of crosslinking reaction. For example, about 0.2% (w/v), about 0.4% (w/v), about 0.6% (w/v), or about 0.8% (w/v) of formaldehyde may be used to crosslink an RNase H enzyme. About 10 minutes of crosslinking reaction using 0.6% formaldehyde may be sufficient to inactivate RNase HII from Pyrococcus furiosus.

The crosslinked RNase HII does not show any measurable endonuclease activity at about 37° C. In some cases, a measurable partial reactivation of the crosslinked RNase HII may occur at a temperature of around 50° C., which is lower than the PCR denaturation temperature. To avoid such unintended reactivation of the enzyme, it may be required to store or keep the modified RNase HII at a temperature lower than 50° C. until its reactivation.

In general, PCR requires heating the amplification composition at each cycle to about 95° C. to denature the double stranded target sequence which will also release the inactivating factor from the RNase H, partially or fully restoring the activity of the enzyme.

RNase H may also be modified by subjecting the enzyme to acylation of lysine residues using an acylating agent, for example, a dicarboxylic acid. Acylation of RNase H may be performed by adding cis-aconitic anhydride to a solution of RNase H in an acylation buffer and incubating the resulting mixture at about 1-20° C. for 5-30 hours. In one embodiment, the acylation may be conducted at around 3-8° C. for 18-24 hours. The type of the acylation buffer is not particularly limited. In an embodiment, the acylation buffer has a pH of between about 7.5 to about 9.0.

The activity of acylated RNase H can be restored by lowering the pH of the amplification composition to about 7.0 or less. For example, when Tris buffer is used as a buffering agent, the composition may be heated to about 95° C., resulting in the lowering of pH from about 8.7 (at 25° C.) to about 6.5 (at 95° C.).

The duration of the heating step in the amplification reaction composition may vary depending on the modified RNase H, the buffer used in the PCR, and the like. However, in general, heating the amplification composition to 95° C. for about 30 seconds—4 minutes is sufficient to restore RNase H activity. In one embodiment, using a commercially available buffer such as Invitrogen AgPath™ buffer (a Tris based buffer (pH 7.6) and one or more non-ionic detergents, full activity of Pyrococcus furiosus RNase HII is restored after about 2 minutes of heating.

RNase H activity may be determined using methods that are well in the art. For example, according to a first method, the unit activity is defined in terms of the acid-solubilization of a certain number of moles of radiolabeled polyadenylic acid in the presence of equimolar polythymidylic acid under defined assay conditions (see Epicentre Hybridase thermostable RNase HI). In the second method, unit activity is defined in terms of a specific increase in the relative fluorescence intensity of a reaction containing equimolar amounts of the probe and a complementary template DNA under defined assay conditions.

### Real-Time Detection of HTLV Target Nucleic Acid Sequences Using a Catacleave Probe

The labeled oligonucleotide probe may be used as a probe for the real-time detection of HTLV target nucleic acid sequences in a sample.

A CataCleave oligonucleotide probe is first synthesized with DNA and RNA sequences that are complimentary to HTLV nucleic acid sequences found within a PCR amplicon comprising a selected HTLV target sequence. In one embodiment, the probe is labeled with a FRET pair, for example, a fluorescein molecule at one end of the probe and a non-fluorescent quencher molecule at the other end.

In certain embodiments cells, such as blood cells, suspected of being infected with HTLV retrovirus are lysed and subjected to the real-time PCR protocols described herein. If HTLV genomic DNA sequences are present in the sample, during the real-time PCR reaction, the labeled probe can hybridize with complementary sequences within the PCR amplicon to form an RNA:DNA heteroduplex that can be cleaved by RNase H. When the RNA sequence portion of the probe is cleaved by the RNase, the two parts of the probe, i.e., a donor and an acceptor, dissociate from a target amplicon into a reaction buffer. As the donor and acceptor separate, FRET is reversed and donor emission can be monitored corresponding to the real-time detection of HTLV target DNA sequences in the sample. Cleavage and dissociation also regenerates a site for further CataCleave™ probe binding on the amplicon. In this way, it is possible for a single amplicon to serve as a target for multiple rounds of probe cleavage until the primer is extended through the CataCleave™ probe binding site.

In certain embodiments, the real-time nucleic acid amplification permits the real-time detection of a single target DNA molecule in less than about 40 PCR amplification cycles.

In certain embodiments, the disclosed methods provide for the detection of one or more HTLV strains, including, but not limited to, HTLV-I, HTLV-II, HTLV-III, and HTLV-IV, but are not limited thereto.

According to an alternative embodiment, total RNA is extracted from cells, reverse transcribed and subjected to real-time Catacleave™-PCR for the detection of HTLV RNA sequences according to the methods described herein.

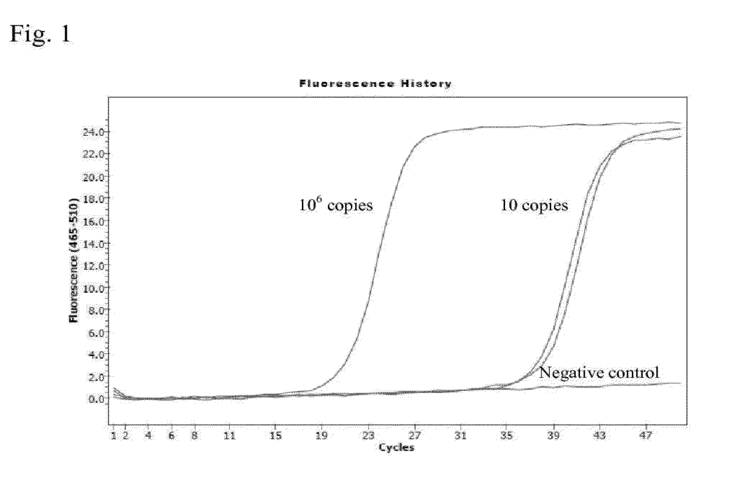

Fluorescence emitted in every cycle of real-time PCR is detected and quantified in real-time using a spectrofluorophotometer, for example, real-time PCR systems 7900, 7500, and 7300 (Applied Biosystems), Mx3000p (Stratagene), Chromo 4 (BioRad), and Roche Lightcycler 480. The real-time PCR device senses the fluorescence marker of the probe of amplified PCR products to show traces as shown in FIG. 1.

The existence of HTLV strains may be identified by calculating a Cₜ value that is the number of cycles when the amount of the amplified PCR products reaches a predetermined level, based on the curve of the fluorescence marker labeled in the probe of the amplified PCR products obtained by the real-time PCR. If the Cₜ value is in the range of 15 to 50, or 20 to 45, it can be concluded that HTLV strains exist. Meanwhile, the Cₜ value may be automatically calculated by a program of the real-time PCR device.

### Kits

The disclosure herein also provides for a kit format which comprises a package unit having one or more reagents for the real-time detection of HTLV nucleic acid sequences sequences in a sample. The kit may also contain one or more of the following items: buffers, instructions, and positive or negative controls. Kits may include containers of reagents mixed together in suitable proportions for performing the methods described herein. Reagent containers preferably contain reagents in unit quantities that obviate measuring steps when performing the subject methods.

Kits may also contain reagents for real-time PCR including, but not limited to, a thermostable polymerase, RNase H, primers selected to amplify selected HTLV nucleic acid sequences and a labeled CataCleave™ oligonucleotide probe that anneals to the real-time PCR product and allow for the detection of the HTLV sequences according to the methodology described herein. Kits may comprise reagents for the simultaneous detection of one or more strains of HTLV. In another embodiment, the kit reagents further comprised reagents for the extraction of genomic DNA or RNA from a biological sample. Kit reagents may also include reagents for reverse transcriptase-PCR analysis where applicable.

### EXAMPLES

The present invention will be described in further detail with reference to the following examples. These examples are for illustrative purposes only and are not intended to limit the scope of the invention.

### Example 1

### Preparation of Primer and Probe for Specific Detection of HTLV-1 or HTLV-2

Primers were first designed to amplify the HTLV-1 or HTLV-2-specific tax gene nucleic acid sequences. The selected nucleotide sequences were identified by analyzing and aligning conserved regions using software Clustal W. Having identified those regions of HTLV-1 and HTLV-2 with conserved regions, the selected sequences were tested for possible cross hybridization against known sequences using a basic local alignment search tool (BLAST). The selected primers with the requisite specificity are shown in Table 1 below. HTLV1 and HTLV2 sequences are available as NCBI accession number AB036370 and NCBI accession number AF326584, respectively.

A CataCleave™ probe that specifically binds to a template of polymerase chain reaction (PCR) was prepared as the probe to PCR products that increase in real-time during real-time PCR. The probe was also designed using the nucleotide sequences of the HTLV tax gene. The 5′ end of the probe was labeled with 6-carboxyfluorescein (FAM) and the 3′ end of the probed was labeled with Black Hole Quencher (Integrated DNA Technologies, Coralville, Iowa).

The nucleotide sequences of the probes used herein are shown in Table 1 below.

TABLE 1

| SEQ | | |
|---|---|---|
| ID | Primer/ | |
| NO: | Probe | Sequence (5′-3′) |
| 1 | HTLV1-G5-F1 | GGCTCAGCTCTACAGTTCCTTA |
| 2 | HTLV1-G5-R1 | AGGAGGGTGGAATGTTGGA |
| 3 | HTLV1-G5-F2 | GCTCTACAGTTCCTTATCCCTCG |
| 4 | HTLV1-G5-R2 | GGCGGGGTAAGGACCTTGAG |
| 5 | HTLV2-G5-F1 | CAGCTCTCCTCTCCAATACC |
| 6 | HTLV2-G5-R1 | GGTGTGCTTTCGCATTGA |
| 7 | HTLV2-G5-F2 | CCTCTCCAATACCTTATCCCTCG |
| 8 | HTLV2-G5-R2 | GGAGGGGTAAGGACCTTGAG |
| 9 | HTLV1/2- | TCCTTCCCrCrArCrCCAGAGAACCT |
| | G5-P | |
| 10 | HTLV1-G6-F1 | GCCAGCCATCTTTAGTACTACAGTCCTC |
| 11 | HTLV1-G6-R1 | CTCATGGTCATTGTCATCTGCCTCT |
| 12 | HTLV1-G6- | CCCATAGTCAGTATCATCTGCCTCT |
| | R1A | |
| 13 | HTLV1-G6-R2 | GCTCATGGTCATTGTCATCTGCCTCT |
| 14 | HTLV1-G6-P1 | TTCAAACCAArGrGrCrCTACCACCCCTCAT |
| 15 | HTLV1-G6-P2 | ACTCTTCCTTTCrArUrArGTTTACATCT |
| 16 | HTLV1-G6-P3 | TTTGAAGAATACrArCrCrAACATCCCCATTT |
| | | CT |
| 17 | HTLV1-G6-P4 | CTCTCACACrGrGrCrCTCATACAGTACTCTT |
| | | CCTT |
| 18 | HTLV1-G6-P5 | CACCAACATCCCrCrArUrUTCTCTACTTTTT |
| | | AAC |
| 19 | HTLV2-G6-F1 | GCAGCCATCTTTAGTAGTTCAGTCCTC |
| 20 | HTLV2-G6-R1 | GCCATTGTCATCCGCCTCT |
| 21 | HTLV2-G6-P1 | TCCAAACCAAArGrCrCrUTCCATCCCTCCT |
| 22 | HTLV2-G6-P2 | ACTCCTCCTrTrCrCrATAACCTTCACCTTC |
| 23 | HTLV2-G6-P3 | TTCGATGAATACrArCrCrAACATCCCTGTCT |
| 24 | HTLV2-G6-P4 | TCTACTCTCTCATCAGCrUrUrArUACAATAC |
| | | TCCTCC |
| 25 | HTLV2-G6-P5 | CTCCTCCTTCCATAArCrCrUrUCACCTTCT |

In Table 1, “r” indicates RNA bases, that is, rG is riboguanosine. Probes were coupled to markers such as FAM (6-carboxyfluorescein) or, BFQ (Black Hole Quencher) for short wavelength emission. In the following Examples, the probes were coupled to FAM at their 5′-end and to BFQ at their 3′-end.

### Example 2

### Method of Detecting HTLV Using Real-time PCR

Plasmid DNA that contains a tax gene from HTLV-1 (NCBI accession number AB036370) was used as detection template.

A mixture including 1 μl of DNA and 24 μl of a PCR mix was used for all real-time PCRs performed herein. The PCR mix included 6.25 μl of a 5× custom PCR buffer solution (Life Tech), 1 μl of 5 μM forward primer G6-F1 (SEQ ID NO: 10), 1 μl of 5 μM reverse primer G6-R1 (SEQ ID NO: 11), 1 μl of 5 μM CataCleave™ probe (SEQ ID NO: 14), 1 μl of dNTP mix (10 μM dGTP, dCTP, dATP, and dTTP), 0.5 μl of 5U/μL Platinum® Taq DNA polymerase (Invitrogen), 0.5 μl of 5U/μL RNase H II, 0.1 μl of 10U/μL uracil-N-glycosylase, and water to bring up to a volume of 25 μL.

Real-time PCR was performed by repeated denaturation at 95° C. for 10 seconds, annealing with the primer and the CataCleave™ probe and reaction with RNase HII at 55° C. for 10 seconds, and elongation at 65° C. for 30 seconds for a total of 60 cycles. The reaction was conducted in a Roche Lightcycler 480. Fluorescence detection of HTLV-1 was monitored in real-time. The results in terms of Cp are shown in Table 2 below.

TABLE 2

| Log (copies/reaction) | Cp |
|---|---|
| 0.7 | 38.01 |
| 1.0 | 37.38 |
| 2.0 | 36.05 |
| 3.0 | 34.99 |
| 5.0 | 28.03 |
| 6.0 | 23.89 |

The results show concentration dependent detection of HTLV-1 with respect to Cp values.

### Example 3

### Detection of HTLV-1 Using CataCleave™ Probe

Real-time PCR of HTLV-1 was performed using a forward primer of SEQ ID 10, a reverse primer of SEQ ID 13, and a CataCleave™ probe of SEQ ID 17. Meanwhile, fluorescence was not detected in a control to which distilled water was added instead of the DNA template. FIG. 1 shows amplification curves of the real-time PCR.

In the experiment, the initial concentration of the template was 10 copies and 10⁶ copies of HTLV1 (tax) plasmid DNA. The results indicate that amplification could be performed with 10 or fewer copies when the real-time PCR was performed using the primer set and CataCleave™ probes. Meanwhile, fluorescence was not detected in a control to which distilled water was added instead of the DNA template.

### Example 4

### Detection of HTLV-2 Using CataCleave™ Probe

Real-time PCR of HTLV-2 was performed using a forward primer of SEQ ID NO: 19, a reverse primer of SEQ ID NO: 20, and a CataCleave™ probe of SEQ ID NO: 23.

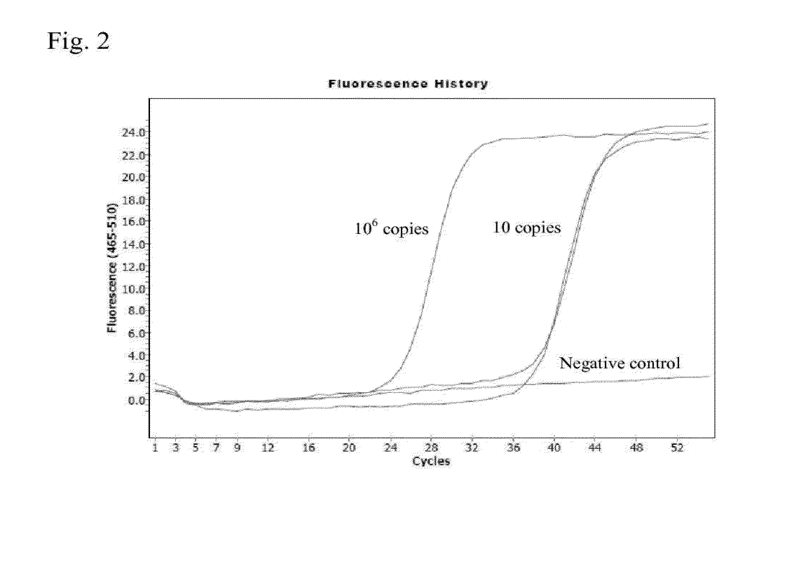

FIG. 2 shows amplification curves obtained by the real-time PCR. Meanwhile, fluorescence was not detected in a control to which distilled water was added instead of the DNA template.

In the experiment, the initial concentration of the template was 10 copies and 10⁵ copies of HTLV2 (tax) plasmid DNA. The results below show that the amplification could be performed with 10 copies or fewer copies when real-time PCR was performed using the primer set and the CataCleave™ probe. Meanwhile, fluorescence was not detected in a control to which distilled water was added instead of the DNA template.

### Example 5

### Simultaneous Detection and Typing of HTLV-1 and HTLV-2 Using CataCleave™ Probe

A multiplex Real-time PCR of HTLV-1 and/or HTLV-2 was performed using two sets of primers and probes. One set includes a forward primer of SEQ ID 10, a reverse primer of SEQ ID 11, a CataCleave™ probe SEQ ID 17, specific to HTLV-1; and the other a forward primer of SEQ ID 19, a reverse primer of SEQ ID 20, and a CataCleave™ probe SEQ ID 25, specific to HTLV-2. HTLV-1 probe (SEQ ID 17) was labeled with a FAM dye, HTLV-2 probe (SEQ ID 25) with a TYE665 dye. Plasmid DNA that contains either HTLV-1 tax or HTLV-2 tax gene was used as template.

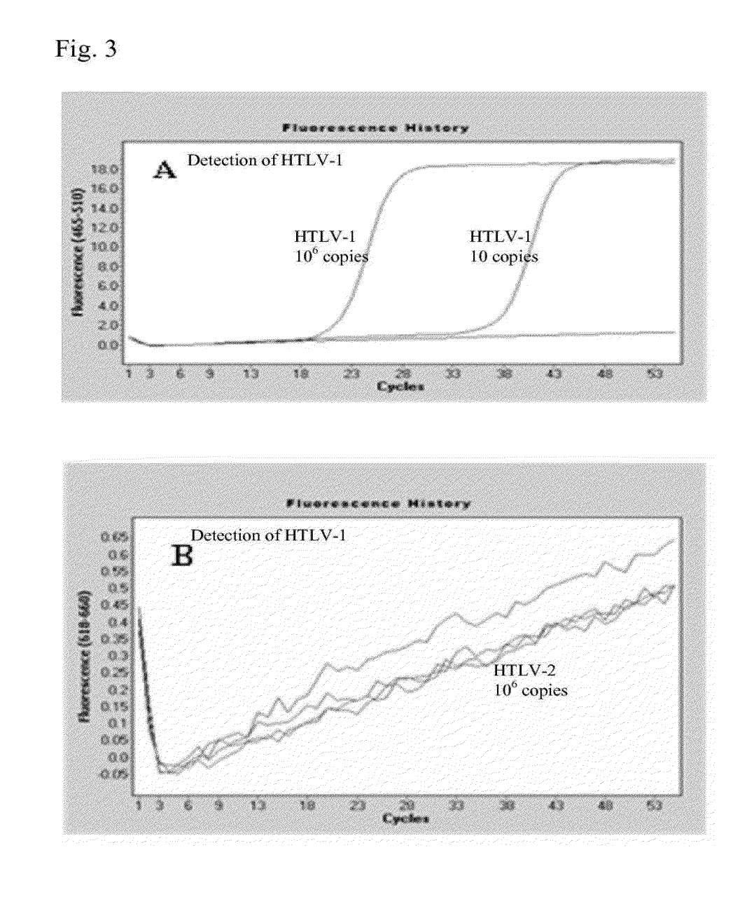

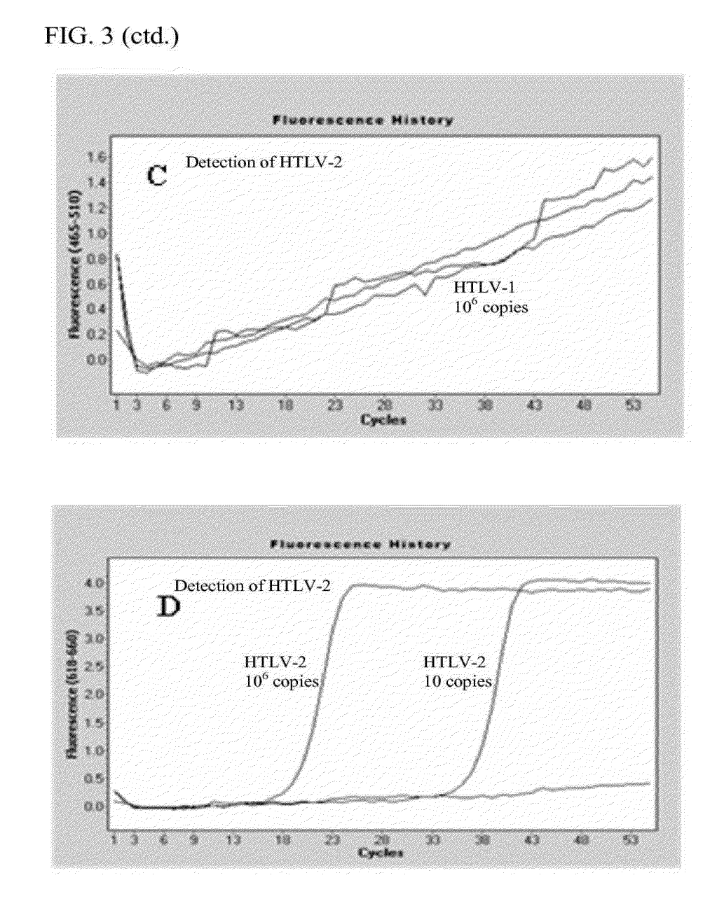

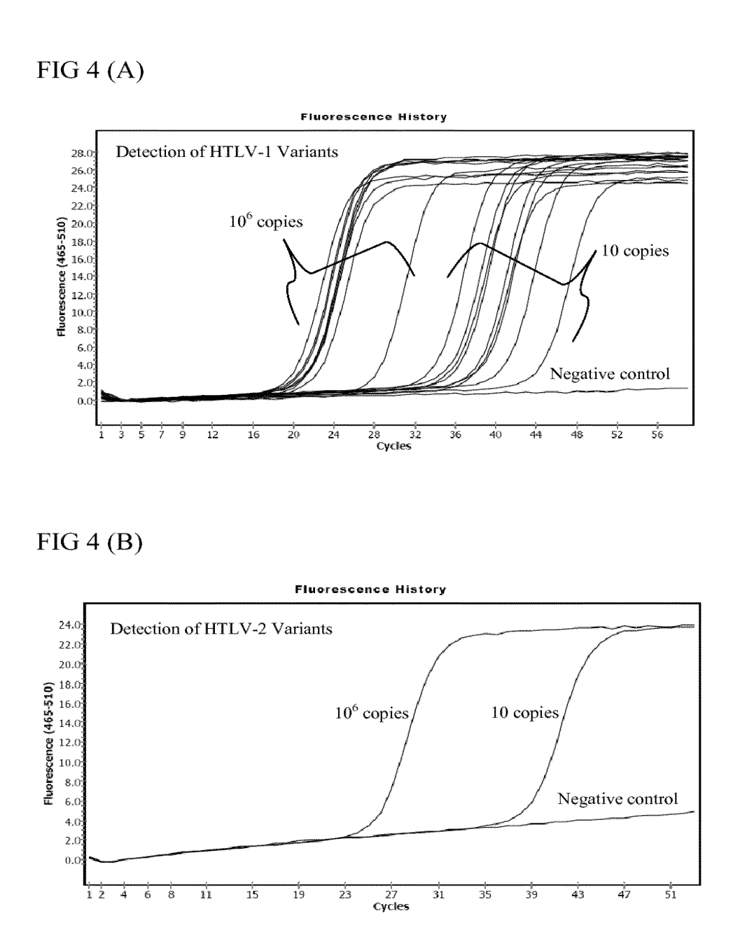

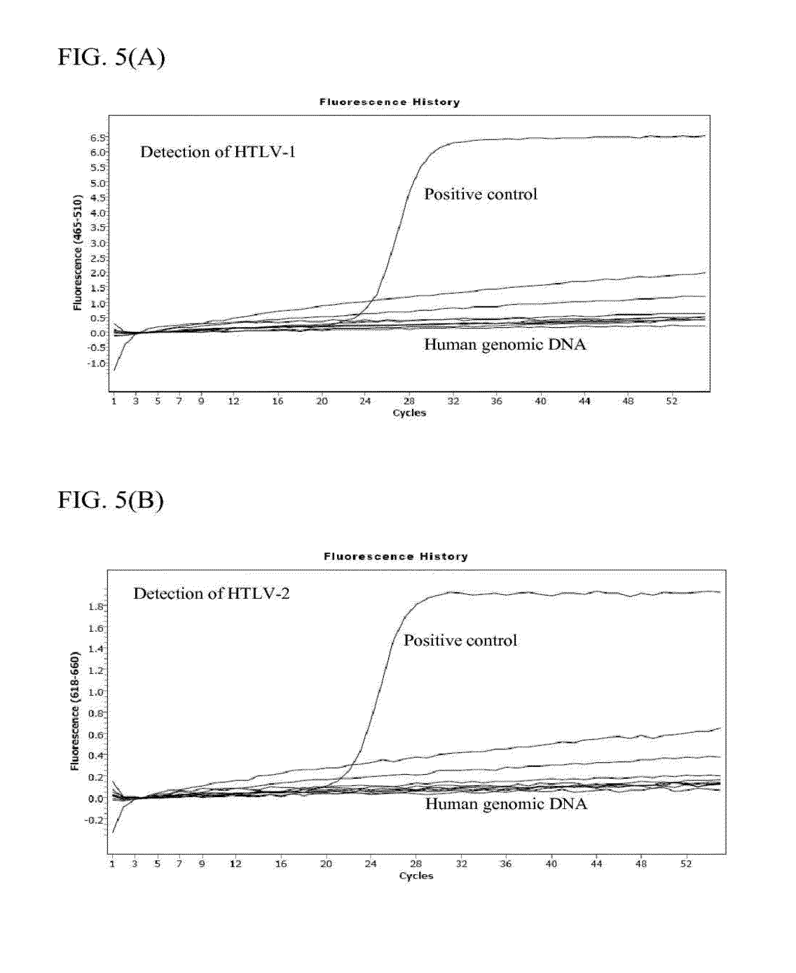

The results are shown in FIGS. 3(A)-3(D), in which

A: Detection of HTLV-1 was 10 and 10⁶ copies with Probe SEQ ID. 17;

B: No fluorescence signals were generated for HTLV-1 by Probe SEQ ID. 25;

C: No fluorescence signals were generated for HTLV-2 by Probe SEQ ID. 17; and

D: Detection HTLV-2 was 10 and 10⁶ copies with Probe SEQ ID. 25.

In the experiment, the initial concentrations of the template were 10 and 10⁶ copies of tax gene in plasmid DNA for both HTLV-1 and HTLV-2. The results show that the multiplex assay not only detects HTLV-1 and HTLV-2 at a concentration down to 10 copies or lower, but also allows discrimination between HTLV-1 and HTLV-2.

### Example 6

### Inclusivity Test

A BLAST search at NCBI showed that a number of HTLV-1 strains have one or more mismatches in the primer and/or probe regions. The accession numbers of HTLV-1 strains examined were AB036380, AB045541, AB045547, AF485380, DQ323882, L36905, L05234, and M67514. The tax genes of these strains were individually incorporated into a cloning vector, and purified. These recombinant plasmid DNA were used as the template for the HTLV-1 inclusivity test. Real-time PCR reactions were conducted as described in EXAMPLE 3.

Similarly, a synthetic DNA fragment that includes all possible mismatches of HTLV-2 strains in the primer/probe regions were synthesized by Integrated DNA Technologies Inc., incorporated into a cloning vector, and purified. This recombinant plasmid DNA was used as the template for the HTLV-2 inclusivity test. Real-time PCR reactions were conducted as described in EXAMPLE 4.

FIG. 4 (A-B) shows that the selected primers and probes can efficiently detect all variants of HTLV-1/2 strains.

### Example 7

### Cross-Reactivity to Human Genomic DNA

A multiplex Real-time PCR of HTLV-1 and HTLV-2 was performed using two sets of primers and probes. One set included a forward primer of SEQ ID 10, a reverse primer of SEQ ID 11, a CataCleave™ probe SEQ ID 17, specific to HTLV-1; and the other a forward primer of SEQ ID 19, a reverse primer of SEQ ID 20, and a CataCleave™ probe SEQ ID 25, specific to HTLV-2. HTLV-1 probe (SEQ ID 17) was labeled with a FAM dye, HTLV-2 probe (SEQ ID 25) with a TYE665 dye. Human genomic DNA (from 10 pg up to 100 ng/reaction) was isolated and used as template.

FIG. 5 (A-B) shows amplification curves obtained during real-time PCR. It was observed that the HTLV detection assay does not cross-react with human genomic DNA. This excludes the possibility of false-positive interference from human genomic DNA which may be co-extracted during nucleic acid isolation.

According to the results mentioned above, HTLV strains can be efficiently detected with high sensitivity and specificity using the primer sets and CataCleave™ probes of the invention. The time and effort for detecting HTLV strains are therefore reduced. Also, as shown in Example 5, the assay allows discrimination between HTLV1 and HTLV2 without any post-PCR handling or treatment.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

1. A primer pair for the PCR detection of HTLV comprising:
at least 10 consecutive nucleotides of a forward amplification primer having a nucleotide sequence selected from the group consisting of SEQ ID NOs: 1, 3, 5, 7, 10 and 19, and
at least 10 consecutive nucleotides of a reverse amplification primer having a nucleotide sequence selected from the group consisting of SEQ ID NOs: 2, 4, 6, 8, 11, 12, 13, 20.

2. A primer probe set for the real-time PCR detection of HTLV strains comprising:
the primer pair of claim 1, and
a probe having a nucleotide sequence selected from the group consisting of SEQ ID NOs: 9, 14, 15, 16, 17, 18, 21, 22, 23, 24 and 25.

3. A kit for the real-time PCR detection of HTLV strains comprising the primer probe set of claim 2.

4. A kit for the real-time PCR detection of HTLV strains comprising one or more of the following primer-probe sets:
a primer-probe set comprising a forward amplification primer comprising at least 10 consecutive nucleotides selected from the nucleotide sequence of SEQ ID NO:1 and a reverse amplification primer comprising at least 10 consecutive nucleotides selected from the nucleotide sequence of SEQ ID NO: 2 and a probe having the nucleotide sequence of SEQ ID NO: 9;
a primer-probe set comprising a forward amplification primer comprising at least 10 consecutive nucleotides selected from the nucleotide sequence of SEQ ID NO: 3 and a reverse amplification primer comprising at least 10 consecutive nucleotides selected from the nucleotide sequence of SEQ ID NO: 4 and a probe having the nucleotide sequence of SEQ ID NO: 9;
a primer-probe set comprising a forward amplification primer comprising at least 10 consecutive nucleotides selected from the nucleotide sequence of SEQ ID NO: 10 and a reverse amplification primer comprising at least 10 consecutive nucleotides selected from the nucleotide sequence of SEQ ID NO: 11, and a probe having the nucleotide sequence of SEQ ID NO: 14, 15, 16, 17 or 18;
a primer-probe set comprising a forward amplification primer comprising at least 10 consecutive nucleotides selected from the nucleotide sequence of SEQ ID NO: 10 and a reverse amplification primer comprising at least 10 consecutive nucleotides selected from the nucleotide sequence of SEQ ID NO: 12, and a probe having the nucleotide sequence of SEQ ID NO: 14, 15, 16, 17 or 18;
a primer-probe set comprising a forward amplification primer comprising at least 10 consecutive nucleotides selected from the nucleotide sequence of SEQ ID NO: 10 and a reverse amplification primer comprising at least 10 consecutive nucleotides selected from the nucleotide sequence of SEQ ID NO: 13, and a probe having the nucleotide sequence of SEQ ID NO: 14, 15, 16, 17 or 18;
a primer-probe set comprising a forward amplification primer comprising at least 10 consecutive nucleotides selected from the nucleotide sequence of SEQ ID NO: 5 and a reverse amplification primer comprising at least 10 consecutive nucleotides selected from the nucleotide sequence of SEQ ID NO: 6 and a probe having the nucleotide sequence of SEQ ID NO: 9;
a primer-probe set comprising a forward amplification primer comprising at least 10 consecutive nucleotides selected from the nucleotide sequence of SEQ ID NO: 7 and a reverse amplification primer comprising at least 10 consecutive nucleotides selected from the nucleotide sequence of SEQ ID NO: 8 and a probe having the nucleotide sequence of SEQ ID NO: 9; and
a primer-probe set comprising a forward amplification primer comprising at least 10 consecutive nucleotides selected from the nucleotide sequence of SEQ ID NO: 19 and a reverse amplification primer comprising at least 10 consecutive nucleotides selected from the nucleotide sequence of SEQ ID NO: 20 and a probe having the nucleotide sequence of SEQ ID NO: 21, 22, 23, 24, 25.

5. The kit of claim 4, further comprising positive internal and negative controls.

6. The kit of claim 4, further comprising uracil-N-glycosylase.

7. The kit of claim 4, wherein the probe is labeled with a FRET pair.

8. The kit of claim 4, wherein the kit further comprises an amplifying polymerase activity.

9. The kit of claim 8, wherein the amplifying polymerase activity is the activity of a thermostable DNA polymerase.

10. The kit of claim 4, wherein the kit further comprises an RNase H activity.

11. The kit of claim 10, wherein the RNase H activity is the enzymatic activity of a thermostable RNase H.

12. The kit of claim 10, wherein the RNase H activity is a hot start RNase H activity.

13. The kit of claim 4, wherein a 5′ end of each probe is labeled with a fluorescent marker selected from the group consisting of FAM, VIC, TET, JOE, HEX, CY3, CY5, ROX, RED610, TEXAS RED, RED670, and NED, and a 3′ end of each probe is labeled with a fluorescence quencher selected from the group consisting of 6-TAMRA, BHQ-1,2,3, and a molecular groove binding non-fluorescence quencher (MGBNFQ).

14. The kit of claim 4, wherein the HTLV strains are selected from the group consisting of HTLV-I, HTLV-II, HTLV-III, and HTLV-IV.

15. A method for the real-time detection of HTLV strains in a sample, comprising the steps of:
a. providing a sample to be tested for the presence of a HTLV gene target DNA;
b. providing a pair of amplification primers that can anneal to the HTML gene target DNA, wherein the pair of amplification primers is selected from a primer-probe set of claim 4;
c. providing a probe of the primer-probe set comprising a detectable label and DNA and RNA nucleic acid sequences that are substantially complimentary to the HTLV target DNA;
d. amplifying a PCR fragment between the forward and reverse amplification primers in the presence of an amplifying polymerase activity, amplification buffer; an RNase H activity and the probe under conditions where the RNA sequences within the probe can form a RNA:DNA heteroduplex with the complimentary DNA sequences in the PCR fragment of the HTLV target DNA; and
e. detecting a real-time increase in the emission of a signal from the label on the probe, wherein the increase in signal indicates the presence of the HTLV target DNA in the sample.

16. The method of claim 15, wherein the real-time increase in the emission of the signal from the label on the probe results from the RNase H cleavage of the heteroduplex formed between the probe and one of the strands of the PCR fragment.

17. The method of claim 15, wherein the probe is labeled with a FRET pair.

18. The method of claim 15, wherein the amplifying polymerase activity is an activity of a thermostable DNA polymerase.

19. The method of claim 15, wherein the RNase H activity is a hot start RNase H activity.

20. The method of claim 15, wherein the HTLV target DNA is a HTLV-I, HTLV-II, HTLV-III, and HTLV-IV target DNA.


SEQUENCE LISTING (30 sequences)

SEQ ID NO: 1 - DNA, 22 bp, Artificial Sequence
  Synthetic oligonucleotide
ggctcagctctacagttcctta

SEQ ID NO: 2 - DNA, 19 bp, Artificial Sequence
  Synthetic oligonucleotide
aggagggtggaatgttgga

SEQ ID NO: 3 - DNA, 23 bp, Artificial Sequence
  Synthetic oligonucleotide
gctctacagttccttatccctcg

SEQ ID NO: 4 - DNA, 20 bp, Artificial Sequence
  Synthetic oligonucleotide
ggcggggtaaggaccttgag

SEQ ID NO: 5 - DNA, 20 bp, Artificial Sequence
  Synthetic oligonucleotide
cagctctcctctccaatacc

SEQ ID NO: 6 - DNA, 18 bp, Artificial Sequence
  Synthetic oligonucleotide
ggtgtgctttcgcattga

SEQ ID NO: 7 - DNA, 23 bp, Artificial Sequence
  Synthetic oligonucleotide
cctctccaataccttatccctcg

SEQ ID NO: 8 - DNA, 20 bp, Artificial Sequence
  Synthetic oligonucleotide
ggaggggtaaggaccttgag

SEQ ID NO: 9 - DNA, 22 bp, Artificial Sequence
  Synthetic oligonucleotide
tccttccccacccagagaacct

SEQ ID NO: 10 - DNA, 28 bp, Artificial Sequence
  Synthetic oligonucleotide
gccagccatctttagtactacagtcctc

SEQ ID NO: 11 - DNA, 25 bp, Artificial Sequence
  Synthetic oligonucleotide
ctcatggtcattgtcatctgcctct

SEQ ID NO: 12 - DNA, 25 bp, Artificial Sequence
  Synthetic oligonucleotide
cccatagtcagtatcatctgcctct

SEQ ID NO: 13 - DNA, 26 bp, Artificial Sequence
  Synthetic oligonucleotide
gctcatggtcattgtcatctgcctct

SEQ ID NO: 14 - DNA, 27 bp, Artificial Sequence
  Synthetic oligonucleotide
ttcaaaccaaggcctaccacccctcat

SEQ ID NO: 15 - DNA, 25 bp, Artificial Sequence
  Synthetic oligonucleotide
actcttcctttcauagtttacatct

SEQ ID NO: 16 - DNA, 30 bp, Artificial Sequence
  Synthetic oligonucleotide
tttgaagaatacaccaacatccccatttct

SEQ ID NO: 17 - DNA, 32 bp, Artificial Sequence
  Synthetic oligonucleotide
ctctcacacggcctcatacagtactcttcctt

SEQ ID NO: 18 - DNA, 31 bp, Artificial Sequence
  Synthetic oligonucleotide
caccaacatccccauutctctactttttaac

SEQ ID NO: 19 - DNA, 27 bp, Artificial Sequence
  Synthetic oligonucleotide
gcagccatctttagtagttcagtcctc

SEQ ID NO: 20 - DNA, 19 bp, Artificial Sequence
  Synthetic oligonucleotide
gccattgtcatccgcctct

SEQ ID NO: 21 - DNA, 27 bp, Artificial Sequence
  Synthetic oligonucleotide
tccaaaccaaagccutccatccctcct

SEQ ID NO: 22 - DNA, 27 bp, Artificial Sequence
  Synthetic oligonucleotide
actcctccttccataaccttcaccttc

SEQ ID NO: 23 - DNA, 28 bp, Artificial Sequence
  Synthetic oligonucleotide
ttcgatgaatacaccaacatccctgtct

SEQ ID NO: 24 - DNA, 34 bp, Artificial Sequence
  Synthetic oligonucleotide
tctactctctcatcagcuuauacaatactcctcc

SEQ ID NO: 25 - DNA, 27 bp, Artificial Sequence
  Synthetic oligonucleotide
ctcctccttccataaccuucaccttct

SEQ ID NO: 26 - protein, 224 aa, Pyrococcus furiosus
MKIGGIDEAGRGPAIGPLVVATVVVDEKNIEKLRNIGVKDSKQLTPHERKNLFSQITSIADDYKIVIVSPEEIDNRSGTMNELEVEKFALALNSLQIKPALIYADAADVDANRFASLIERRLNYKAKIIAEHKADAKYPVVSAASILAKVVRDEEIEKLKKQYGDFGSGYPSDPKTKKWLEEYYKKHNSFPPIVRRTWETVRKIEESIKAKKSQLTLDKFFKKP

SEQ ID NO: 27 - protein, 16 aa, Artificial Sequence
  Synthetic polypeptide
GIDEAGRGPAIGPLVV

SEQ ID NO: 28 - protein, 12 aa, Artificial Sequence
  Synthetic polypeptide
LRNIGVKDSKQL

SEQ ID NO: 29 - protein, 19 aa, Artificial Sequence
  Synthetic polypeptide
HKADAKYPVVSAASILAKV

SEQ ID NO: 30 - protein, 16 aa, Artificial Sequence
  Synthetic polypeptide
KLKKQYGDFGSGYPSD